SEQUENCE LISTING (49 sequences)

SEQ ID NO: 1 - protein, 19 aa, Artificial Sequence
  Amino acid sequence derived from a polypeptide component of a human immune system checkpoint
FMTYWHLLNAFTVTVPKDL

SEQ ID NO: 2 - protein, 28 aa, Artificial Sequence
  Amino acid sequence derived from a polypeptide component of a human immune system checkpoint
RTHLVILGAILLCLGVALTFIFRLRKGR

SEQ ID NO: 3 - protein, 23 aa, Artificial Sequence
  Amino acid sequence derived from a polypeptide component of a human immune system checkpoint
VILGAILLCLGVALTFIFRLRKG

SEQ ID NO: 4 - protein, 9 aa, Artificial Sequence
  Amino acid sequence derived from a polypeptide component of a human immune system checkpoint
LLNAFTVTV

SEQ ID NO: 5 - protein, 9 aa, Artificial Sequence
  Amino acid sequence derived from a polypeptide component of a human immune system checkpoint
ILLCLGVAL

SEQ ID NO: 6 - protein, 9 aa, Artificial Sequence
  Amino acid sequence derived from a polypeptide component of a human immune system checkpoint
ILGAILLCL

SEQ ID NO: 7 - protein, 9 aa, Artificial Sequence
  Amino acid sequence derived from a polypeptide component of a human immune system checkpoint
ALQITDVKL

SEQ ID NO: 8 - protein, 9 aa, Artificial Sequence
  Amino acid sequence derived from a polypeptide component of a human immune system checkpoint
KLFNVTSTL

SEQ ID NO: 9 - protein, 9 aa, Artificial Sequence
  Amino acid sequence derived from a polypeptide component of a human immune system checkpoint
RLLKDQLSL

SEQ ID NO: 10 - protein, 9 aa, Artificial Sequence
  Amino acid sequence derived from a polypeptide component of a human immune system checkpoint
QLSLGNAAL

SEQ ID NO: 11 - protein, 9 aa, Artificial Sequence
  Amino acid sequence derived from a polypeptide component of a human immune system checkpoint
KINQRILVV

SEQ ID NO: 12 - protein, 9 aa, Artificial Sequence
  Amino acid sequence derived from a polypeptide component of a human immune system checkpoint
HLVILGAIL

SEQ ID NO: 13 - protein, 9 aa, Artificial Sequence
  Amino acid sequence derived from a polypeptide component of a human immune system checkpoint
RINTTTNEI

SEQ ID NO: 14 - protein, 9 aa, Artificial Sequence
  Amino acid sequence derived from a polypeptide component of a human immune system checkpoint
CLGVALTFI

SEQ ID NO: 15 - protein, 9 aa, Artificial Sequence
  Amino acid sequence derived from a polypeptide component of a human immune system checkpoint
QLDLAALIV

SEQ ID NO: 16 - protein, 9 aa, Artificial Sequence
  Amino acid sequence derived from a polypeptide component of a human immune system checkpoint
SLGNAALQI

SEQ ID NO: 17 - protein, 10 aa, Artificial Sequence
  Amino acid sequence derived from a polypeptide component of a human immune system checkpoint
VILGAILLCL

SEQ ID NO: 18 - protein, 10 aa, Artificial Sequence
  Amino acid sequence derived from a polypeptide component of a human immune system checkpoint
HTAELVIPEL

SEQ ID NO: 19 - protein, 10 aa, Artificial Sequence
  Amino acid sequence derived from a polypeptide component of a human immune system checkpoint
FIFMTYWHLL

SEQ ID NO: 20 - protein, 10 aa, Artificial Sequence
  Amino acid sequence derived from a polypeptide component of a human immune system checkpoint
VIWTSSDHQV

SEQ ID NO: 21 - protein, 21 aa, Artificial Sequence
  Amino acid sequence derived from a polypeptide component of a human immune system checkpoint
DTLLKALLEIASCLEKALQVF

SEQ ID NO: 22 - protein, 9 aa, Artificial Sequence
  Amino acid sequence derived from a polypeptide component of a human immune system checkpoint
ALLEIASCL

SEQ ID NO: 23 - protein, 9 aa, Artificial Sequence
  Amino acid sequence derived from a polypeptide component of a human immune system checkpoint
QLRERVEKL

SEQ ID NO: 24 - protein, 9 aa, Artificial Sequence
  Amino acid sequence derived from a polypeptide component of a human immune system checkpoint
FLVSLLVEI

SEQ ID NO: 25 - protein, 9 aa, Artificial Sequence
  Amino acid sequence derived from a polypeptide component of a human immune system checkpoint
TLLKALLEI

SEQ ID NO: 26 - protein, 9 aa, Artificial Sequence
  Amino acid sequence derived from a polypeptide component of a human immune system checkpoint
FIAKHLPDL

SEQ ID NO: 27 - protein, 9 aa, Artificial Sequence
  Amino acid sequence derived from a polypeptide component of a human immune system checkpoint
VLSKGDAGL

SEQ ID NO: 28 - protein, 9 aa, Artificial Sequence
  Amino acid sequence derived from a polypeptide component of a human immune system checkpoint
DLMNFLKTV

SEQ ID NO: 29 - protein, 9 aa, Artificial Sequence
  Amino acid sequence derived from a polypeptide component of a human immune system checkpoint
VLLGIQQTA

SEQ ID NO: 30 - protein, 9 aa, Artificial Sequence
  Amino acid sequence derived from a polypeptide component of a human immune system checkpoint
KVLPRNIAV

SEQ ID NO: 31 - protein, 10 aa, Artificial Sequence
  Amino acid sequences derived from polypeptide components of human immune system checkpoints
KLNMLSIDHL

SEQ ID NO: 32 - protein, 10 aa, Artificial Sequence
  Amino acid sequence derived from a polypeptide component of a human immune system checkpoint
SLRSYHLQIV

SEQ ID NO: 33 - protein, 10 aa, Artificial Sequence
  Amino acid sequence derived from a polypeptide component of a human immune system checkpoint
RLLENKIGVL

SEQ ID NO: 34 - protein, 10 aa, Artificial Sequence
  Amino acid sequence derived from a polypeptide component of a human immune system checkpoint
TLLELVEAWL

SEQ ID NO: 35 - protein, 10 aa, Artificial Sequence
  Amino acid sequence derived from a polypeptide component of a human immune system checkpoint
FIITHQAYEL

SEQ ID NO: 36 - protein, 9 aa, Artificial Sequence
  Amino acid sequence derived from a polypeptide component of a human immune system checkpoint
LIYGNYLHL

SEQ ID NO: 37 - protein, 10 aa, Artificial Sequence
  Amino acid sequence derived from a polypeptide component of a human immune system checkpoint
KLLVQQFSIL

SEQ ID NO: 38 - protein, 10 aa, Artificial Sequence
  Amino acid sequence derived from a polypeptide component of a human immune system checkpoint
KIHDEHLFII

SEQ ID NO: 39 - protein, 10 aa, Artificial Sequence
  Amino acid sequence derived from a polypeptide component of a human immune system checkpoint
LLKSEQEKTL

SEQ ID NO: 40 - protein, 9 aa, Artificial Sequence
  Amino acid sequence derived from a polypeptide component of a human immune system checkpoint
QLLTSLMDI

SEQ ID NO: 41 - protein, 9 aa, Artificial Sequence
  Amino acid sequence derived from a polypeptide component of a human immune system checkpoint
QILWELDSV

SEQ ID NO: 42 - protein, 9 aa, Artificial Sequence
  Amino acid sequence derived from a polypeptide component of a human immune system checkpoint
SILETMTAL

SEQ ID NO: 43 - protein, 9 aa, Artificial Sequence
  Amino acid sequence derived from a polypeptide component of a human immune system checkpoint
LLSKGERRL

SEQ ID NO: 44 - protein, 9 aa, Artificial Sequence
  Amino acid sequence derived from a polypeptide component of a human immune system checkpoint
DLFNLSTYL

SEQ ID NO: 45 - protein, 10 aa, Artificial Sequence
  Amino acid sequence derived from a polypeptide component of a human immune system checkpoint
KLEKNITRGL

SEQ ID NO: 46 - protein, 10 aa, Artificial Sequence
  Amino acid sequence derived from a polypeptide component of a human immune system checkpoint
LIPRHWIPKM

SEQ ID NO: 47 - protein, 10 aa, Artificial Sequence
  Amino acid sequence derived from a polypeptide component of a human immune system checkpoint
KMNPTIHKFL

SEQ ID NO: 48 - protein, 20 aa, Artificial Sequence
  Amino acid sequence derived from a polypeptide component of a human immune system checkpoint
VSVILKLLVQQFSILETMTA

SEQ ID NO: 49 - protein, 20 aa, Artificial Sequence
  Amino acid sequence derived from a polypeptide component of a human immune system checkpoint
RFQVPFQLLTSLMDIDSLMT

(19) United States
(12) Patent Application Publication
(10) Pub. No.: US 2020/0299401 A1
(43) Pub. Date: Sep. 24, 2020

(54) ENHANCEMENT OF ANTIBODY-DEPENDENT CELL-MEDIATED CYTOTOXICITY (ADCC)

(71) Applicant: IO Biotech ApS, Copenhagen (DK)

(72) Inventor: Mads Hald ANDERSEN, Nærum (DK)

(21) Appl. No.: 16/765,654

(22) PCT Filed: Nov. 23, 2018
(86) PCT No.: PCT/EP2018/082427

§ 371 Date: May 20, 2020

(30) Foreign Application Priority Data
Nov. 24, 2017 (GB) 1719561.1
Apr. 23, 2018 (GB) 1806575.5

(51) Int. Cl. C07K 16/28; A61P 35/00

(52) U.S. Cl. CPC C07K 16/2896; C07K 2317/734; C07K 2317/732; A61P 35/00

(57) ABSTRACT

The present invention relates to a method for increasing the therapeutic benefit of an antibody to a subject. The improved benefit is typically mediated by an increase in the antibody-dependent cell-mediated cytotoxicity (ADCC) effect of the antibody. The method comprises (a) administering to said subject an immunotherapeutic composition comprising a component of an immune system checkpoint, or an immunogenic fragment of said component; and (b) also administering said antibody to the subject. The increase in therapeutic benefit to the subject may be in respect of any disease for which the said antibody has a prophylactic or therapeutic effect. The disease may be cancer. The invention also relates to said immunotherapeutic composition and said antibody, and to kits comprising same.

### FIELD OF THE INVENTION

The present invention relates to a method for increasing the therapeutic benefit of an antibody to a subject. The improved benefit is typically mediated by an increase in the antibody-dependent cell-mediated cytotoxicity (ADCC) effect of the antibody. The method comprises (a) administering to said subject an immunotherapeutic composition comprising a component of an immune system checkpoint, or an immunogenic fragment of said component; and (b) also administering said antibody to the subject. The increase in therapeutic benefit to the subject may be in respect of any disease for which the said antibody has a prophylactic or therapeutic effect. The disease may be cancer.

The invention also relates to said immunotherapeutic composition and said antibody, and to kits comprising same.

### BACKGROUND TO THE INVENTION

The therapeutic efficacy of some antibodies depends on the capacity of the antibody to recognize its target (e.g. an antigen on a tumour cell) and induce cytotoxicity via a network of immune effector cells. This process is referred to as antibody-dependent cell-mediated cytotoxicity (ADCC). It is triggered by the interaction of the fragment crystallizable (Fc) portion of the antibody with Fc receptors (FcRs) on a complex network of effector cells including natural killer (NK) cells, macrophages, γδ T cells, and dendritic cells. Although previously thought to be a relatively simple process by which effector immune cells lyse antibody-coated target cells through the release of cytotoxic molecules like perforin and granzyme, ADCC is now known to be a complex process that co-ordination and linked regulation of the above-mentioned immune cell-types.

Several antibodies, including rituximab, trastuzumab, alemtuzumab, cetuximab, panitumumab, and ofatumumab, have become standard of care for the treatment of both solid tumors and hematological malignancies. However many patients may experience a low or sometimes no therapeutic effect from such antibody treatments. Accordingly, there exists a need for methods which augment the ADCC of such antibodies.

### SUMMARY OF THE INVENTION

The inventors have surprisingly shown that an immunotherapeutic composition comprising a component of an immune system checkpoint, or an immunogenic fragment thereof, may augment the ADCC effect of an antibody that is administered to a subject as a therapy. Immunotherapeutic compositions of this type have previously been shown to promote CD4+ and CD8+ T cell responses to a checkpoint component, leading to an inhibition of the effect of the said checkpoint as well as direct T cell mediated killing of cancer cells expressing the checkpoint component. See, for example, WO 2009/143843, WO2013/056716, WO/2016/041560 and PCT/EP2017/055093—these documents, and specifically the individually disclosed peptide sequences and any compositions comprising them, are herein incorporated by reference in their entirety. Such compositions have been described as immunomodulatory vaccines because of their influence on checkpoint activity. However, an effect on immune effector mechanisms which are not directly mediated by CD4+ and CD8+ cell responses has not previously been described. In particular, such compositions have not previously been recognised as having any effect on ADCC.

The present invention relates to a method for increasing the therapeutic benefit of an antibody to a subject. The method comprises (a) administering to said subject an immunotherapeutic composition comprising a component of an immune system checkpoint, or an immunogenic fragment of said component; and (b) also administering said antibody to the subject. Steps (a) and (b) may be conducted simultaneously, separately or sequentially.

The antibody may be any antibody that is administered to a subject as a treatment for a disease, and the increase in therapeutic benefit is typically in respect of the said disease. The method may therefore also be described as a method for treating the said disease with an immunotherapeutic composition and an antibody, which method is more effective than administering only the antibody. The antibody has preferably been demonstrated to have a therapeutic effect which is at least partially mediated by antibody-dependent cell-mediated cytotoxicity (ADCC). The antibody is typically an anti-cancer antibody. An anti-cancer antibody means any antibody which is indicated for the treatment of a cancer. Such an antibody typically binds specifically to an antigen expressed on the surface of a cancer cell.

Thus the present invention also provides a method for the prevention or treatment of cancer in a subject, the method comprising administering to said subject:
(i) an immunotherapeutic composition comprising a component of an immune system checkpoint, or an immunogenic fragment of said component; and
(ii) an anti-cancer antibody.

The present invention also provides a kit comprising said immunotherapeutic composition and said antibody.

### BRIEF DESCRIPTION OF THE SEQUENCE LISTING

SEQ ID NOs: 1-49 are amino acid sequences derived from polypeptide components of immune system checkpoints. They are shown in full in Table 1.

### BRIEF DESCRIPTION OF THE FIGURES

FIG. 1: Example of a leukapheresis product which shows an IFN-γ response to stimulation with PDL1 long1 (lower panel), which is associated with a potentiation of ADCC by daratumumab (upper panel).

FIG. 2: Example of a healthy donor buffy coat which shows an IFN-γ response to stimulation with PDL1 long1 (lower panel), which is associated with a potentiation of ADCC by daratumumab (upper panel).

FIG. 3: Example of a healthy donor buffy coat without IFN-γ response to stimulation with PDL1 long1 (lower panel). No potentiation of ADCC by daratumumab is seen (upper panel).

FIG. 4(A): IFN-γ ELISPOT assays of responses to stimulation with the peptide IO103 (FMTYWHLLNAFTVTVPKDL) (black bars) or without peptide stimulation (white bars) in leukapheresis products from patients with multiple myeloma. Leukapheresis samples from 20 patients were tested. Experiments were carried out in triplicate (n=10) or duplicate (n=10) when cell recovery was poor. The cells from patient 18 died. *indicates a triplicate with significant DFRx1. **indicates a triplicate with significant DFRx2 as described by Moodie et al 2012. (*) indicates a duplicate with a p-value<0.05 (student's t-test). Cell counts per well: Patient 1: 4.0×10⁵; Patient 2: 2.5×10⁵; Patient 3: 4.0×10⁵; Patient 4: 2.8×10⁵; Patient 5: 4.0×10⁵; Patient 6: 3.6×10⁵; Patient 7: 2.7×10⁵; Patient 8: 3.7×10⁵; Patient 9: 3.4×10⁵; Patient 10: 6.0×10⁵; Patient 11: 3.0×10⁵; Patient 12: 4.0×10⁵; Patient 13: 3.0×10⁵; Patient 14: 2.3×10⁵; Patient 15: 3.0×10⁵; Patient 16: 4.0×10⁵; Patient 17: 4.0×10⁵; Patient 19: 4.0×10⁵; Patient 20: 6.0×10⁵. The figure depicts means with standard error of the mean (SEM).

FIG. 4(B): Example of a T-cell response against the IO103 peptide in the leukapheresis product from a myeloma patient (Patient 9).

FIG. 4(C): Results showing that previously prepared HLA-A2-restricted PD-L1-specific CTLs lyse T2 cells pulsed with PD-L1 peptide as well as the PD-L1-positive, HLA-A2-positive multiple myeloma cell line U266. Killing was measured by a standard ⁵¹Cr-release assay. Treatment with IFN-γ upregulates PD-L1 and mediates a tendency towards increased killing of the myeloma cell line (means with standard deviation (SD)).

FIG. 5: Representative results showing that stimulation of patient leukapheresis or healthy donor PBMCs augments the ADCC effect of daratumumab against CD38-positive myeloma. Leukapheresis products from different MM patients (leukapharesis 1, 9, 11 and 20) or PBMCs from healthy donors (HD-361, HD-382) were stimulated with either IO103 or scrambled control peptide as shown. On day 2, IL-2 was added at 120 U/ml. On day 7, the leukapheresis products or PBMCs were washed, counted, and used as effector cells at the ratios shown in a ⁵¹Cr-release cytotoxic assay+/−daratumumab against the CD38-positive cell line RPMI-8226 (means with SD).

FIG. 6: Schematic diagram of the study design for the Examples.

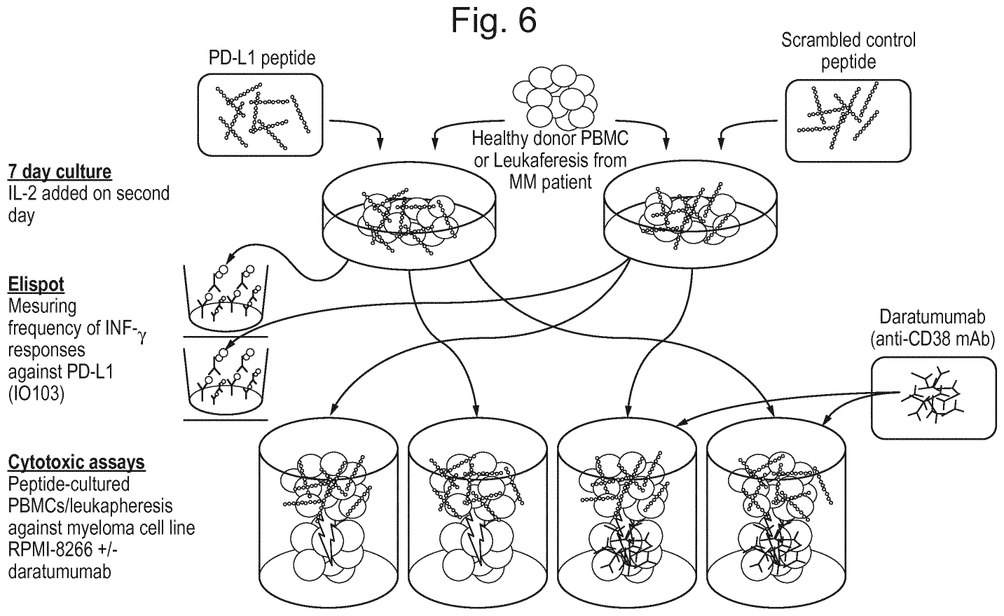

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

The terms “patient” and “subject” are used interchangeably and typically refer to a human.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

All publications, patents and patent applications cited herein, whether supra or infra, are hereby incorporated by reference in their entirety.

### Immune System Checkpoint

The term “immune system checkpoint” is used herein to refer to any molecular interaction which alters the balance in favour of inhibition of the immune effector responses. That is, a molecular interaction which, when it occurs, negatively regulates the activation of an immune effector cell. Such an interaction might be direct, such as the interaction between a ligand and a cell surface receptor which transmits an inhibitory signal into an effector cell. Or it might be indirect, such as the blocking or inhibition of an interaction between a ligand and a cell surface receptor which would otherwise transmit an activatory signal into the effector cell, or an interaction which promotes the upregulation of an inhibitory molecule or cell, or the depletion by an enzyme of a metabolite required by the effector cell, or any combination thereof.

Examples of immune system checkpoints include:
a) The interaction between PD1 and PDL1 and/or PD1 and PDL2;
b) The interaction between Indoleamine 2,3-dioxygenase (IDO1) or tryptophan 2,3-dioxygenase (TDO) and their substrate (tryptophan)
c) The interaction between Arginase 1 (Arg1) or Arginase 2 (Arg2) and their substrate (arginine)
d) The interaction between CTLA4 and CD86 and/or CTLA4 and CD80;
e) The interaction between B7-H3 and/or B7-H4 and their respective ligands;
f) The interaction between HVEM and BTLA;
g) The interaction between GAL9 and TIM3;
h) The interaction between MHC class I or II and LAG3; and
i) The interaction between MHC class I or II and KIR

Checkpoint (a), namely the interaction between PD1 and either of its ligands PDL1 and PDL2, is a preferred checkpoint for the purposes of the present invention. PD1 is expressed on effector T cells. Engagement with either ligand results in a signal which downregulates activation. The ligands are expressed by some tumours. PDL1 in particular is expressed by many solid tumours, including melanoma. These tumours may therefore down regulate immune mediated anti-tumour effects through activation of the inhibitory PD1 receptors on T cells. By blocking the interaction between PD1 and one or both of its ligands, a checkpoint of the immune response may be removed, leading to augmented anti-tumour T cell responses. The inventors have shown that targeting the PD1-PDL1 interaction by administering an immunotherapeutic composition comprising immunogenic fragments of PDL1 unexpectedly also leads to augmented ADCC activity. The same can be expected if the PD1-PDL2 interaction is targeted in this way, and similar results may be expected by targeting any one of the checkpoints listed above. Therefore PD1 and its ligands are examples of components of an immune system checkpoint which may preferably be targeted in the method of the invention by means of a suitable immunotherapeutic composition. PDL1 is a particularly interesting target because it interacts with CD80 as well as PD1.

Another preferred checkpoint for the purposes of the present invention is checkpoint (b), namely the interaction between IDO1 or TDO and their substrate. This checkpoint is the metabolic pathway in cells of the immune system requiring the essential amino acid tryptophan. A lack of tryptophan results in the general suppression of effector T cell functions and promotes the conversion of naïve T cells into regulatory (i.e. immunosuppressive) T cells (Tregs). This may also suppress ADCC activity. The protein IDO1 is upregulated in cells of many tumours and is responsible for degrading the level of tryptophan. IDO1 is an enzyme that catalyzes the conversion of L-tryptophan to N-formylkynurenine and is thus the first and rate limiting enzyme of tryptophan catabolism through the Kynurenine pathway. TDO catalyses the same step. Therefore, IDO1 and TDO are components of an immune system checkpoint and each may preferably be targeted in the method of the invention by means of a suitable immunotherapeutic composition.

Another preferred checkpoint for the purposes of the present invention is checkpoint (c), namely the interaction between Arg1 or Arg2 and their substrate. The arginases are enzymes that catalyse a reaction which converts the amino acid L-arginine into L-ornithine and urea. This depletes the microenvironment of arginine and leads to a suppression of tumor-specific cytotoxic T-cell responses. Increased Arginase1 activity has been detected in the cancer cells of patients with breast, lung, colon or prostate cancer. Therefore, Arg1 and Arg2 are components of an immune system checkpoint and each may preferably be targeted in the method of the invention by means of a suitable immunotherapeutic composition.

Another preferred checkpoint for the purposes of the present invention is checkpoint (d), namely the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. This may also lead to augmented ADCC activity. Therefore CTLA4 and its ligands are examples of components of an immune system checkpoint which may preferably be targeted in the method of the invention by means of a suitable immunotherapeutic composition.

### Immunotherapeutic Composition

An immunotherapeutic composition of the invention results in an immune response against a component of an immune system checkpoint, preferably a checkpoint as described in the preceding section. The component is typically a polypeptide. Thus, the immunotherapeutic composition may comprise said component or may comprise an immunogenic fragment thereof. An “immunogenic fragment” is used herein to mean a polypeptide which is shorter than the said component of an immune system checkpoint, but which is capable of eliciting an immune response to said component.

The ability of a fragment to elicit an immune response (“immunogenicity”) to a component of an immune system checkpoint may be assessed by any suitable method. Typically, the fragment will be capable of inducing proliferation and/or cytokine release in vitro in T cells specific for the said component, wherein said cells may be present in a sample of lymphocytes taken from a donor, such as a healthy individual or preferably a cancer patient. Proliferation and/or cytokine release may be assessed by any suitable method, including ELISA and ELISPOT. Exemplary methods are described in the Examples. Preferably, the fragment induces proliferation of component-specific T cells and/or induces the release of interferon gamma from such cells.

In order to induce proliferation and/or cytokine release in T cells specific for the said component, the fragment must be capable of binding to an MHC molecule such that it is presented to a T cell. In other words, the fragment comprises or consists of at least one MHC binding epitope of the said component. Said epitope may be an MHC Class I binding epitope or an MHC Class II binding epitope. It is particularly preferred if the fragment comprises more than one MHC binding epitope, each of which said epitopes binds to an MHC molecule expressed from a different HLA-allele, thereby increasing the breadth of coverage of subjects taken from an outbred human population.

MHC binding may be evaluated by any suitable method including the use of in silico methods. Preferred methods include competitive inhibition assays wherein binding is measured relative to a reference peptide. The reference peptide is typically a peptide which is known to be a strong binder for a given MHC molecule. In such an assay, a peptide is a weak binder for a given HLA molecule if it has an IC50 more than 100 fold lower than the reference peptide for the given HLA molecule. A peptide is a moderate binder is it has an IC50 more than 20 fold lower but less than a 100 fold lower than the reference peptide for the given HLA molecule. A peptide is a strong binder if it has an IC50 less than 20 fold lower than the reference peptide for the given HLA molecule.

A fragment comprising an MHC Class I epitope preferably binds to a MHC Class I HLA species selected from the group consisting of HLA-A1, HLA-A2, HLA-A3, HLA-A11 and HLA-A24, more preferably HLA-A3 or HLA-A2. Alternatively the fragment may bind to a MHC Class I HLA-B species selected from the group consisting of HLA-B7, HLA-B35, HLA-B44, HLA-B8, HLA-B15, HLA-B27 and HLA-B51.

A fragment comprising an MHC Class II epitope preferably binds to a MHC Class II HLA species selected from the group consisting of HLA-DPA-1, HLA-DPB-1, HLA-DQA1, HLA-DQB1, HLA-DRA, HLA-DRB and all alleles in these groups and HLA-DM, HLA-DO.

The immunotherapeutic composition may comprise one immunogenic fragment of a component of an immune system checkpoint, or may comprise a combination of two or more such fragments, each interacting specifically with at least one different HLA molecule so as to cover a larger proportion of the target population. Thus, as examples, the composition may contain a combination of a peptide restricted by a HLA-A molecule and a peptide restricted by a HLA-B molecule, e.g. including those HLA-A and HLA-B molecules that correspond to the prevalence of HLA phenotypes in the target population, such as e.g. HLA-A2 and HLA-B35. Additionally, the composition may comprise a peptide restricted by an HLA-C molecule.

A preferred immunotherapeutic composition of the invention preferably results in an immune response against at least one of the immune system checkpoints described in the previous section. In other words, the method of the invention preferably comprises administering an immunotherapeutic composition which results in an immune response against at least one said checkpoint. The immunotherapeutic composition may thus alternatively be described as a vaccine against one or more checkpoint components, and more precisely an immunomodulatory vaccine against said one or more checkpoint components.

The immunotherapeutic composition of the invention may comprise the checkpoint component or an immunogenic fragment thereof. The said fragment may consist of at least 8, preferably at least 9 consecutive amino acids of the said component. The said fragment may consist of up to 50 consecutive amino acids of the said component, up to 40 consecutive amino acids of the said component, up to 30 consecutive amino acids of the said component, or up to 25 consecutive amino acids of the said component. Thus, the fragment may comprise or consist of 8 to 50, 8 to 40, 8 to 30, 8 to 25, 9 to 50, 9 to 40, 9 to 30, or 9 to 25 consecutive amino acids of the said component. The fragment preferably comprises or consists of 9 to 30 consecutive amino acids of the said component. The said consecutive amino acids of the fragment preferably comprise or consist of any one of the sequences provided in Table 1. Thus, the said fragment may comprise or consist of any one of the sequences provided in Table 1. The said fragment preferably comprises or consists of any one of the sequences marked “*” in Table 1. The said fragment most preferably comprises or consists of any one of the sequences marked “#” in Table 1.

TABLE 1

| | Source | | SEQ |
|---|---|---|---|
| Name | protein | | ID |
| IO103 | PDL1 | FMTYWHLLNAFTVTV | 1 |
| | | PKDL*# | |
| IO104.1 | PDL1 | RTHLVILGAILLCLG | 2 |
| | | VALTFIFRLRKGR*# | |
| IO104 | PDL1 | VILGAILLCLGVALT | 3 |
| | | FIFRLRKG* | |
| POL101 | PDL1 | LLNAFTVTV* | 4 |
| POL102 | PDL1 | ILLCLGVAL* | 5 |
| POL103 | PDL1 | ILGAILLCL* | 6 |
| POL104 | PDL1 | ALQITDVKL | 7 |
| POL105 | PDL1 | KLFNVTSTL | 8 |
| POL106 | PDL1 | RLLKDQLSL | 9 |
| POL107 | PDL1 | QLSLGNAAL | 10 |
| POL108 | PDL1 | KINQRILVV | 11 |
| POL109 | PDL1 | HLVILGAIL* | 12 |
| POL110 | PDL1 | RINTTTNEI | 13 |
| POL111 | PDL1 | CLGVALTFI* | 14 |
| POL112 | PDL1 | QLDLAALIV | 15 |
| POL113 | PDL1 | SLGNAALQI | 16 |
| POL114 | PDL1 | VILGAILLCL* | 17 |
| POL115 | PDL1 | HTAELVIPEL | 18 |
| POL116 | PDL1 | FIFMTYWHLL* | 19 |
| POL117 | PDL1 | VIWTSSDHQV | 20 |
| IO102 | IDO | DTLLKALLEIASCLE | 21 |
| | | KALQVF*# | |
| IO101 | IDO | ALLEIASCL* | 22 |
| IOx1 | IDO | QLRERVEKL | 23 |
| IOx2 | IDO | FLVSLLVEI | 24 |
| IOx3 | IDO | TLLKALLEI | 25 |
| IOx4 | IDO | FIAKHLPDL | 26 |
| IOx6 | IDO | VLSKGDAGL | 27 |
| IOx7 | IDO | DLMNFLKTV | 28 |
| IOx8 | IDO | VLLGIQQTA | 29 |
| IOx9 | IDO | KVLPRNIAV | 30 |
| IOx10 | IDO | KLNMLSIDHL | 31 |
| IOx11 | IDO | SLRSYHLQIV | 32 |
| TDO1 | TDO | RLLENKIGVL | 33 |
| TDO2 | TDO | TLLELVEAWL | 34 |
| TDO3 | TDO | FIITHQAYEL | 35 |
| TDO4 | TDO | LIYGNYLHL | 36 |
| TDO5 | TDO | KLLVQQFSIL | 37 |
| TDO6 | TDO | KIHDEHLFII | 38 |
| TDO7 | TDO | LLKSEQEKTL | 39 |
| TDO8 | TDO | QLLTSLMDI | 40 |
| TDO9 | TDO | QILWELDSV | 41 |
| TDO10 | TDO | SILETMTAL | 42 |
| TDO11 | TDO | LLSKGERRL | 43 |
| TDO12 | TDO | DLFNLSTYL | 44 |
| TDO13 | TDO | KLEKNITRGL | 45 |
| TDO14 | TDO | LIPRHWIPKM | 46 |
| TDO15 | TDO | KMNPTIHKFL | 47 |
| TDOlong1 | TDO | VSVILKLLVQQFSIL | 48 |
| | | ETMTA* | |
| TDOlong2 | TDO | RFQVPFQLLTSLMDI | 49 |
| | | DSLMT* | |

An immunotherapeutic composition may preferably comprise an adjuvant and/or a carrier or excipient. Adjuvants are any substance whose admixture into the composition increases or otherwise modifies the immune response elicited by the composition. Adjuvants, broadly defined, are substances which promote immune responses. Adjuvants may also preferably have a depot effect, in that they also result in a slow and sustained release of an active agent from the administration site. A general discussion of adjuvants is provided in Goding, Monoclonal Antibodies: Principles & Practice (2nd edition, 1986) at pages 61-63.

Adjuvants may be selected from the group consisting of: AlK(SO4)2, AlNa(SO4)2, AlNH4 (SO4), silica, alum, Al(OH)3, Ca3 (PO4)2, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Various saponin extracts have also been suggested to be useful as adjuvants in immunogenic compositions. Granulocyte-macrophage colony stimulating factor (GM-CSF) may also be used as an adjuvant.

Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. GM-CSF and Imidazochinilines are also examples of preferred adjuvants.

The adjuvant is most preferably a Montanide ISA adjuvant. The Montanide ISA adjuvant is preferably Montanide ISA 51 or Montanide ISA 720.

In Goding, Monoclonal Antibodies: Principles & Practice (2nd edition, 1986) at pages 61-63 it is also noted that, when an antigen of interest is of low molecular weight, or is poorly immunogenic, coupling to an immunogenic carrier is recommended. A polypeptide or fragment of an immunotherapeutic composition of the invention may be coupled to a carrier. A carrier may be present independently of an adjuvant. The function of a carrier can be, for example, to increase the molecular weight of a polypeptide fragment in order to increase activity or immunogenicity, to confer stability, to increase the biological activity, or to increase serum half-life. Furthermore, a carrier may aid in presenting the polypeptide or fragment thereof to T-cells. Thus, in the immunogenic composition, the polypeptide or fragment thereof may be associated with a carrier such as those set out below.

The carrier may be any suitable carrier known to a person skilled in the art, for example a protein or an antigen presenting cell, such as a dendritic cell (DC). Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively the carrier protein may be tetanus toxoid or diphtheria toxoid. Alternatively, the carrier may be a dextran such as sepharose. The carrier must be physiologically acceptable to humans and safe.

The immunotherapeutic composition may optionally comprise a pharmaceutically acceptable excipient. The excipient must be ‘acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

The immunotherapeutic composition may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e. g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Antibodies

An antibody for use in the method of the invention may be any antibody that is administered to a subject as a treatment for a disease, and the increase in therapeutic benefit is typically in respect of the said disease. The antibody has preferably been demonstrated to have a therapeutic effect which is at least partially mediated by antibody-dependent cell-mediated cytotoxicity (ADCC). The antibody is typically an anti-cancer antibody. An anti-cancer antibody means any antibody which is indicated for the treatment of a cancer. Such an antibody typically binds specifically to an antigen expressed on the surface of a cancer cell. The antigen may be described as tumour antigen.

The cancer may be Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, Carcinoid tumor, gastrointestinal, Carcinoma of unknown primary, Central nervous system lymphoma, Cerebellar astrocytoma, Cerebral astrocytoma/Malignant glioma, Cervical cancer, Chronic lymphocytic leukemia, Chronic myelogenous leukemia Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) cancer, Gastrointestinal Carcinoid Tumor, Gastrointestinal stromal tumor (GIST), Germ cell tumor: extracranial, extragonadal, or ovarian, Gestational trophoblastic tumor, Glioma of the brain stem, Glioma, Childhood Cerebral Astrocytoma, Glioma, Childhood Visual Pathway and Hypothalamic, Gastric carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liposarcoma, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Macroglobulinemia, Waldenström, Malignant Fibrous Histiocytoma of Bone/Osteosarcoma, Medulloblastoma, Melanoma, Melanoma, Intraocular (Eye), Merkel Cell Carcinoma, Mesothelioma, Adult Malignant, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, Salivary gland cancer, Sarcoma, Ewing family of tumors, Kaposi Sarcoma, Sarcoma, soft tissue, Sarcoma, uterine, Sézary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma, Squamous neck cancer with occult primary, metastatic, Stomach cancer, Supratentorial primitive neuroectodermal tumor, T-Cell lymphoma, cutaneous—see Mycosis Fungoides and Sézary syndrome, Testicular cancer, Throat cancer, Thymoma, Thymoma and Thymic carcinoma, Thyroid cancer, Thyroid cancer, Transitional cell cancer of the renal pelvis and ureter, Trophoblastic tumor, Ureter and renal pelvis, transitional cell cancer Urethral cancer, Uterine cancer, endometrial, Uterine sarcoma, Vaginal cancer, Visual pathway and hypothalamic glioma, Vulvar cancer, Waldenström macroglobulinemia and Wilms tumor (kidney cancer).

The cancer is preferably multiple myeloma, prostate cancer, breast cancer, bladder cancer, colon cancer, rectal cancer, pancreatic cancer, ovarian cancer, lung cancer, cervical cancer, endometrial cancer, kidney (renal cell) cancer, oesophageal cancer, thyroid cancer, skin cancer, lymphoma, melanoma or leukemia.

Antigens targets of interest for an antibody for use in the method include CD2, CD3, CD19, CD20, CD22, CD25, CD30, CD32, CD33, CD40, CD52, CD54, CD56, CD64, CD70, CD74, CD79, CD80, CD86, CD105, CD138, CD174, CD205, CD227, CD326, CD340, MUC16, GPNMB, PSMA, Cripto, ED-B, TMEFF2, EphA2, EphB2, FAP, av integrin, Mesothelin, EGFR, TAG-72, GD2, CA1X, 5T4, α4β7 integrin, Her2. Other targets are cytokines, such as interleukins IL-1 through IL-13, tumour necrosis factors α & β, interferons α, β and γ, tumour growth factor Beta (TGF-β), colony stimulating factor (CSF) and granulocyte monocyte colony stimulating factor (GMCSF). See Human Cytokines: Handbook for Basic & Clinical Research (Aggrawal et al. eds., Blackwell Scientific, Boston, Mass. 1991). Other targets are hormones, enzymes, and intracellular and intercellular messengers, such as, adenyl cyclase, guanyl cyclase, and phospholipase C. Other targets of interest are leukocyte antigens, such as CD20, and CD33.

The antibody may be Abagovomab, Abciximab, Actoxumab, Adalimumab, Adecatumumab, Afelimomab, Afutuzumab, Alacizumab pegol, ALD518, Alemtuzumab, Alirocumab, Altumomab pentetate, Amatuximab, Anatumomab mafenatox, Anrukinzumab, Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Elotuzumab Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obinutuzumab, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tralokinumab, Trastuzumab, TRBS07, Tregalizumab, Tremelimumab Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab Vesencumab, Visilizumab, Volociximab, Vorsetuzumab mafodotin, Votumumab, Zalutumumab, Zanolimumab, Zatuximab, Ziralimumab or Zolimomab aritox.

Preferred antibodies include Natalizumab, Vedolizumab, Belimumab, Atacicept, Alefacept, Otelixizumab, Teplizumab, Rituximab, Ofatumumab, Ocrelizumab, Epratuzumab, Alemtuzumab, Abatacept, Eculizumab, Omalizumab, Canakinumab, Meplizumab, Reslizumab, Tocilizumab, Ustekinumab, Briakinumab, Etanercept, Inlfliximab, Adalimumab, Certolizumab pegol, Golimumab, Trastuzumab, Gemtuzumab, Ozogamicin, Ibritumomab, Tiuxetan, Tostitumomab, Cetuximab, Bevacizumab, Panitumumab, Denosumab, Ipilimumab, Brentuximab and Vedotin.

Particularly preferred antibodies that may be used in the method of the invention include: daratumumab, nivolumab, pembrolizumab, avelumab, rituximab, trastuzumab, pertuzumab, alemtuzumab, cetuximab, panitumumab, tositumomab and ofatumumab. Daratumumab is especially preferred.

### Methods for Improving Therapeutic Benefit of an Antibody

The present invention provides a method for improving the benefit to a subject of an antibody, preferably an antibody as described in the preceding section. The improved benefit is typically mediated by an increase in the ADCC effect of the antibody. The level of an ADCC response in a subject may be determined by any suitable technique. Such techniques may include testing a sample taken from the subject for ADCC activity, for example using a Cr51 release assay as described in the Examples, or using suitable fluorescent labels such as calcein or europium, or an enzymatic assay which detects the activity of enzymes released from lysed cells.

The method comprises (a) administering to said subject an immunotherapeutic composition comprising a component of an immune system checkpoint, or an immunogenic fragment of said component; and (b) also administering said antibody to the subject. Steps (a) and (b) may be conducted simultaneously, separately or sequentially.

Thus the present invention also provides a method for the prevention or treatment of a disease in a subject, the method comprising administering to said subject:
(i) an immunotherapeutic composition comprising a component of an immune system checkpoint, or an immunogenic fragment of said component; and
(ii) an antibody for the treatment of the said disease.

The present invention also provides an immunotherapeutic composition comprising a component of an immune system checkpoint, or an immunogenic fragment of said component, for use in a method for treating a disease in a subject, wherein the method comprises (i) administering said composition to the subject and (ii) administering an antibody for the treatment of the said disease to said subject.

The present invention also provides an antibody for use in a method for treating a disease in a subject, wherein the antibody is suitable for the treatment of the said disease and wherein the method comprises (i) administering said antibody to the subject and (ii) administering an immunotherapeutic composition comprising a component of an immune system checkpoint, or an immunogenic fragment of said component, to said subject.

The present invention also provides for the use of an immunotherapeutic composition comprising a component of an immune system checkpoint, or an immunogenic fragment of said component, in the manufacture of a medicament for the treatment of a disease, wherein the medicament is for use in a method comprising (i) administering said medicament to the subject and (ii) administering an antibody for the treatment of the said disease to said subject.

The present invention also provides for the use of an antibody, in the manufacture of a medicament for the treatment of a disease, wherein the antibody is suitable for the treatment of the said disease and the medicament is for use in a method comprising (i) administering said medicament to the subject and (ii) administering an immunotherapeutic composition comprising a component of an immune system checkpoint, or an immunogenic fragment of said component, to said subject.

In each of the above embodiments the disease is typically cancer.

### Administration Regimen

In a method of the invention, the immunotherapeutic composition and antibody are each administered to the subject in a therapeutically effective amount. By a “therapeutically effective amount” of a substance, it is meant that a given substance is administered to a subject in an amount sufficient to cure, alleviate or partially arrest the disease or one or more of its symptoms. Such therapeutic treatment may result in a decrease in severity of disease symptoms, or an increase in frequency or duration of symptom-free periods. Such treatment may result in a reduction in the volume of a solid tumour.

In order to prevent disease, the immunotherapeutic composition and antibody are each administered to the subject in a prophylactically effective amount. By “prophylactically effective amount” of a substance, it is meant that a given substance is administered to a subject in an amount sufficient to prevent occurrence or recurrence of one or more of symptoms associated with disease for an extended period.

Effective amounts for a given purpose and a given composition or agent will depend on the severity of the disease as well as the weight and general state of the subject, and may be readily determined by the physician.

The immunotherapeutic composition and antibody may be administered simultaneously or sequentially, in any order. The appropriate administration routes and doses for each may be determined by a physician, and the composition and agent formulated accordingly.

The immunotherapeutic composition is typically administered via a parenteral route, typically by injection. Administration may preferably be via a subcutaneous, intradermal, intramuscular, or intratumoral route. The injection site may be pre-treated, for example with imiquimod or a similar topical adjuvant to enhance immunogenicity. The total amount of polypeptide present as active agent in a single dose of an immunotherapeutic composition of the invention will typically be in the range of 10 μg to 1000 μg, preferably 10 μg to 150 μg.

Antibodies are typically administered as a systemic infusion, for example intravenously. Appropriate doses for antibodies may be determined by a physician. Appropriate doses for antibodies are typically proportionate to the body weight of the subject.

A typical regimen for the method of the invention will involve multiple, independent administrations of both the immunotherapeutic composition and antibody. Each may be independently administered on more than one occasion, such as two, three, four, five, six, seven or more times. The immunotherapeutic composition in particular may provide an increased benefit if it is administered on more than one occasion, since repeat doses may boost the resulting immune response. Individual administrations of composition or antibody may be separated by an appropriate interval determined by a physician, but the interval will typically be 1-2 weeks. The interval between administrations will typically be shorter at the beginning of a course of treatment, and will increase towards the end of a course of treatment.

An exemplary administration regimen comprises administration of an antibody at, for example a dose of 3 milligram per kilogram of body weight, every three weeks for a total of around four series, with an immunotherapeutic composition (typically including an adjuvant) also administered subcutaneously on the back of the arm or front of the thigh, alternating between the right and the left side. Administration of the immunotherapeutic composition may be initiated concomitantly with the first series of antibody, with a total of around 7 doses of composition delivered; first weekly for a total of four and thereafter three additional doses biweekly.

Another exemplary administration regimen comprises treating subjects every second week (induction) for 2.5 months and thereafter monthly (maintenance) with an immunotherapeutic composition (typically including adjuvant) administered subcutaneously. Imiquimod ointment (Aldara, Meda AS, www.meda.se) may optionally be administered 8 hours before administration of the composition and the skin covered by a patch until administration in the same area of the skin.

### Kits

The invention also provides a kit suitable for use in a method of the invention, the kit containing an effective amount of an immunotherapeutic composition. The kits of the invention may additionally comprise one or more other reagents or instruments which enable any of the embodiments mentioned above to be carried out. Such reagents or instruments include one or more of the following: a therapeutically effective amount of an antibody, suitable buffer(s) (aqueous solutions), means to administer the agent to a subject as an intravenous infusion (such as a vessel or an instrument comprising a needle). Reagents may be present in the kit in a dry state such that a fluid sample resuspends the reagents. The kit may also, optionally, comprise instructions to enable the kit to be used in the method of the invention or details regarding which patients the method may be used for.

The invention is illustrated by the following Examples.

### EXAMPLE 1

### Introduction

The presence of PDL1 specific T-cells has previously been demonstrated in cancer patients, and to a lesser extent in healthy subjects. In vitro stimulation of PBMCs with PDL1 long1 (IO103) peptide boosts the activity of PDL1-specific T cells, leading to cytotoxic killing of various cancer cells, including myeloma cells. A phase I clinical trial is underway in patients with MM.

Similarly, patients with MM frequently harbour PDL1-specific T cells. Leukapheresis products from these patients expand IFNγ-secreting PDL1 specific T cells (detected by ELISPOT) after in vitro stimulation in the presence of PDL1 long1 peptide (IO103).

The following studies were conducted to assess whether vaccination with PDL1 long1 can also boost the ADCC effect of a therapeutic antibody, which would provide a clear indication that a combination of an immunotherapeutic composition and a therapeutic antibody could be highly beneficial. Since such compositions are typically extremely well tolerated (without >grade 1 toxicity), the proposed potentiation will likely not increase toxicity of a therapeutic antibody such as daratumumab.

Daratumumab was selected as an exemplary therapeutic antibody for use in the study because it is already FDA- and EMA-approved, currently as a second line treatment of multiple myeloma (MM) when given in combination with lenalidomide and dexamethasone. It is an IgG1 mAb against CD38 with potent antibody-dependent cell-mediated cytotoxicity (ADCC) and complement dependent cytotoxicity (CDC) activities.

### Materials and Methods

### Cells

Cryopreserved leukapheresis products from 20 multiple myeloma patients and cryopreserved buffy coats from healthy donors were thawed into hematopoietic cell medium (Lonza X-vivo) for use in these assays. After thawing, cells were rested for two hours and counted. The leukapheresis products were obtained with appropriate ethics committee approval. All cells were cryopreserved at −150° C. in FBS+10% DMSO in 1.8 ml cryovials.

### In Vitro Stimulation With an Immunogenic Fragment of PDL1 (IO103)

Day one: 24 well plates were prepared with 6 wells of cells from leukapheresis products (˜6×10⁶ cells per well in hematopoietic cell medium) and 6 wells of cells from healthy donor buffy coats (˜7×10⁶ cells per well hematopoietic cell medium). 3 wells of each cell type were incubated with the PDL1 peptide fragment IO103 (10 μM) and 3 wells were incubated with a scrambled control peptide (10 μM). The peptide IO103 may also be referred to as PDLong1.

Day two: IL-2 120 U/ml (Proleukin, Novartis) was added to all wells.

Day eight: Each well was divided in two to provide effector cells for testing in a Chromium⁵¹-release assay for CTL mediated cytotoxicity, except for a small sub-sample retained for ELIPSOT testing. Half of the cells from each well had daratumumab (Janssen Oncology) (0.5 μg/ml ) added and half did not. See further details of the cytotoxicity assay below.

ELISPOT testing was conducted as described in Example 1 of WO2013/056716 to determine whether the cells release IFN-g in response to stimulation with the IO103 peptide versus control. Positive results in this assay were taken as indicative of the presence of PDL1 specific immune responses in the donor. Most leukapharesis products had PDL1 specific immune responses. Some healthy donors also had PDL1 specific immune responses.

### Chromium⁵¹-Release Cytotoxicity Assay Against Multiple Myeloma Tumour Cell Line Target Cells

RPMI-8226 cells (from ATCC: Multiple myeloma cell line: HLA-A2 negative, CD38-positive, PDL1 positive; may also be referred to in this Example as RPMI-8266) were obtained for use as target cells.

The target cells were prepared in advance by spinning down 0.5×10⁶ RPMI-8266 cells and discarding the supernatant leaving approximately 100 μl. These cells were then incubated with ⁵¹Cr at 37° C. for 60-90 minutes before washing twice and in RPMI-1640+10% FCS and resuspending also in RPMI-1640+10% FCS.

The washed target cells were plated out in 96-well plates with the different effector cells from the in vitro stimulation, +/−daratumumab at various E:T ratios. All were then incubated at 37° C. for 4 h, before 100 μl of medium was aspirated and ⁵¹Cr release counted in a gamma counter (Perkin Elmer).

Maximum ⁵¹Cr release was determined in separate wells by addition of 100 μl 10% Triton X-100 to of target cells only. Spontaneous release was determined in separate wells by the addition of 100 μl R10 to target cells only.

Specific lysis was calculated using the following formula: ((experimental release−spontaneous release)/(maximum release−spontaneous release))×100.

### Results

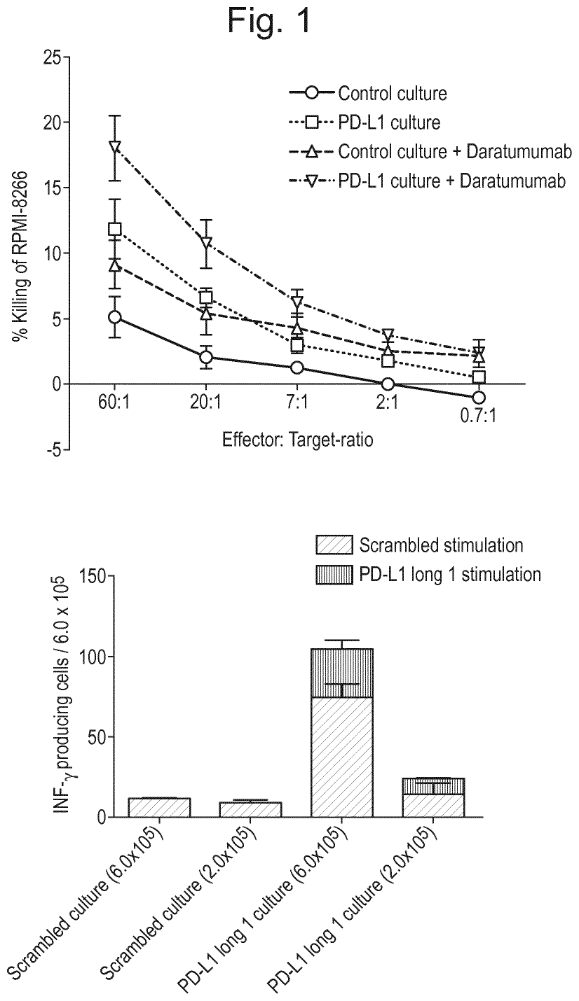

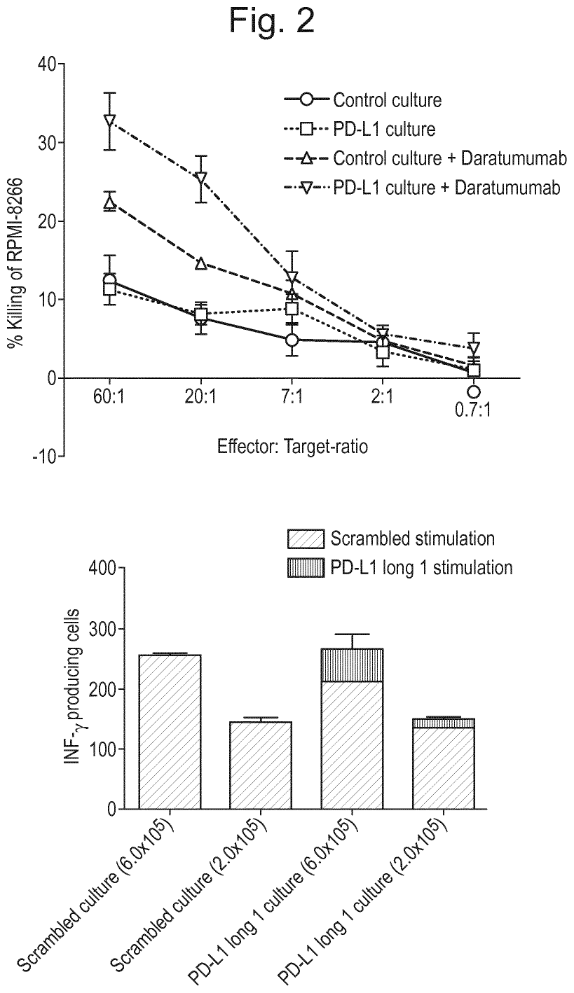

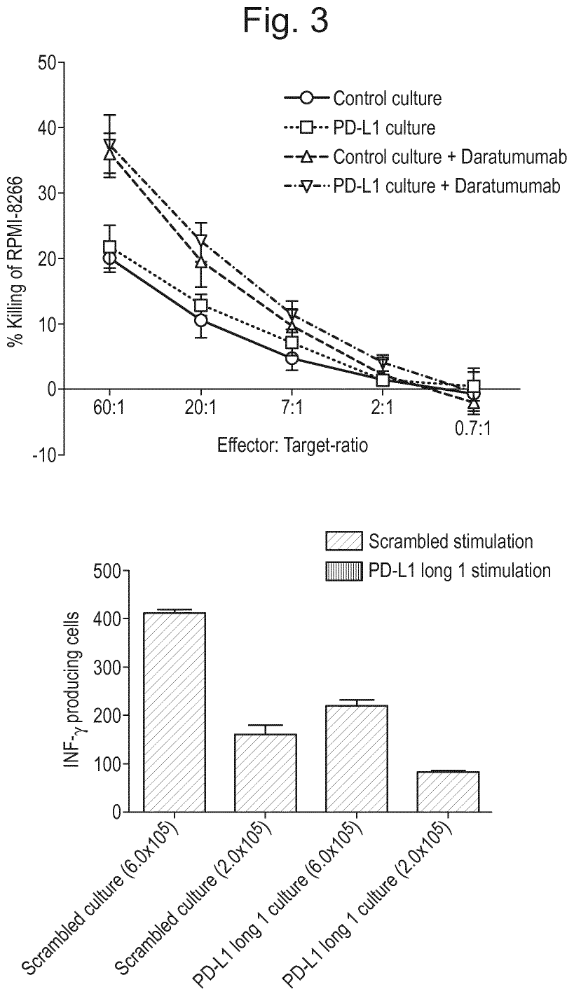

FIGS. 1, 2 and 3 provide representative results from independent experiments. Summarizing the findings:
1. MM-leukapharesis products typically demonstrated measurable PDL1 specific responses in the IFNγ ELISPOT assay following stimulation with the PDL1 peptide IO103. See representative results in FIG. 1, lower panel.
Leukapharesis product cells stimulated with IO103 and tested without daratumumab typically demonstrated a significantly greater cytotoxic killing effect in the Cr51 assay than cells stimulated with control peptide and tested without daratumab. The level of cytotoxic killing was comparable to that achieved with control peptide stimulated cells plus daratumamb, indicating that stimulation with IO103 alone induces a good cytotoxic response. This may be because the MM patients harbor MM-specific T-cells, leading to relative large degree of T-cell killing of target cells in the absence of any antibody. However, the greatest level of cytotoxic killing was seen with cells stimulated with IO103 and tested with daratumamb, indicating that the IO103 stimulation also potentiates the ADCC activity of daratumamb. See representative results in FIG. 1, upper panel.
2. PBMCs from healthy donors sometimes also demonstrate measurable PDL1 specific responses in the IFNγ ELISPOT assay following stimulation with the PDL1 peptide IO103. See representative results in FIG. 2, lower panel.
The cells from these donors also showed an enhancement of the ADCC effect of daratumumab following stimulation with IO103 versus control peptide. In the absence of daratumumab, cells stimulated with either peptide did not demonstrate good levels of killing. The daratumumab-potentiation effect is therefore a more predominant effect in these healthy donors as compared to the MM patients, suggesting that the healthy donors do not harbor any MM-specific T cells. See representative results in FIG. 2, upper panel.
3. PBMCs from healthy donors sometimes do not demonstrate measurable PDL1 specific responses in the IFNγ ELISPOT assay following stimulation with the PDL1 peptide IO103. See representative results in FIG. 3, lower panel.
The cells from these donors showed no enhancement of the ADCC effect of daratumumab following stimulation with IO103 versus control peptide. In the absence of daratumuab, cells stimulated with either peptide did not demonstrate good levels of killing. See representative results in FIG. 3, upper panel.

### Conclusion

This study demonstrates potentiation of the daratumumab-mediated killing of cancer cells by stimulating effector cells with an immunogenic fragment of PDL1 (IO103). This suggests that a vaccination with IO103 used in combination with daratumumab would provide a simple method to potentiate the effect of daratumumab. This also provides proof of principle that an immunotherapeutic composition/immunomodulatory vaccine may potentiate the ADCC effect of therapeutic antibodies in general.

### EXAMPLE 2

Additional experiments were conducted on further samples of leukapharesis products obtained from MM patients and healthy donors (see methods as in Example 1). The myeloma cell lines U266 and RPMI-8226 cell lines were obtained from American Type Culture Collection (ATCC) and cultured according to the manufacturer's instructions. Daratumumab and IL2 were obtained as in Example 1.

### Materials and Methods

### ELISPOT

ELISPOT assays used the IO103 peptide as in Example 1 (FMTYWHLLNAFTVTVPKDL; SEQ ID NO: 1). Interferon gamma (IFN-γ)-ELISPOT against the peptide was performed on leukapheresis products after stimulation with the peptide for 7 days in vitro. The ELISPOT procedure was conducted as in Example 1. ELISPOT assays were performed according to the guidelines of the Association for Cancer Immunotherapy (CIP). When possible, the samples were run in triplicates, and peptide-stimulated cells and negative controls were compared using the non-parametric distribution free resampling (DFR) test as described in Moodie et al 2012 (Response Determination Criteria for ELISPOT: Toward a Standard that Can Be Applied Across Laboratories. In: Methods in Molecular Biology. Vol 792; 2012:185-196). When sample viability allowed duplicate tests, the Student's t-test was used to compare results.

### Cytotoxic Assays

The cytotoxicity assay was performed as in Example 1. Briefly, HLA-A2-positive CTLs that were specific to PD-L1 were thawed, rested over-night and used as effector cells against ⁵¹Cr-labeled HLA-A2-positive U266 myeloma cells in different effector to target (E:T)-cell ratios.

### ADCC

ADCC was assessed in ⁵¹Cr-release cytotoxic assays using the myeloma cell line RPMI-8226 as in Example 1. Briefly, Leukapheresis products or healthy donor PBMCs were thawed on day one, rested for two hours, and counted. The cells were stimulated in triplicate with PD-L1 long1 (10 μM) or control scrambled peptide (10 μM). On day two, 120 U/ml interleukin-2 was added to the wells. On day eight, samples in each well were split in half and used as effector cells against RPMI-8226+/−0.5 μg/ml daratumumab in a ⁵¹Cr-release assay. Leukapheresis products and the cell line RPMI-8226 did not have matching HLA-types.

### Results and Discussion

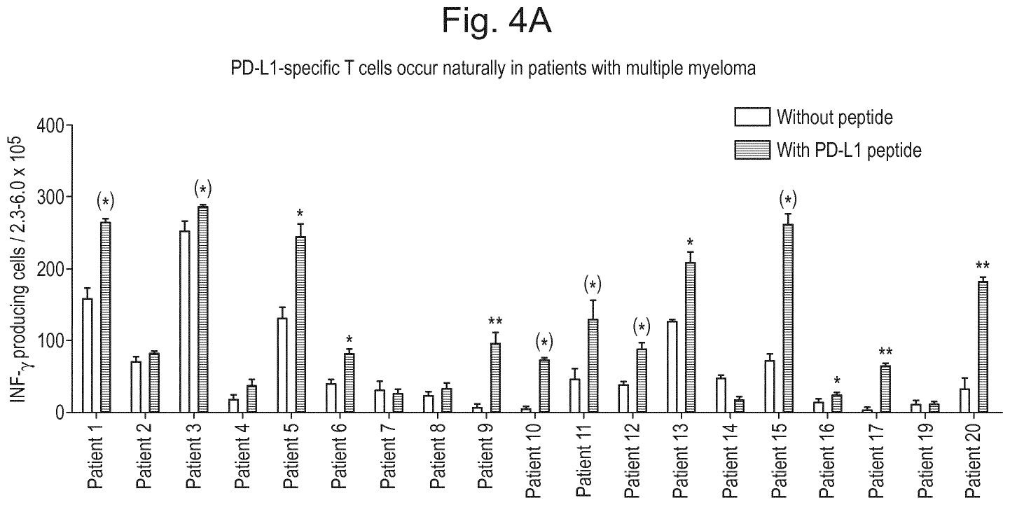

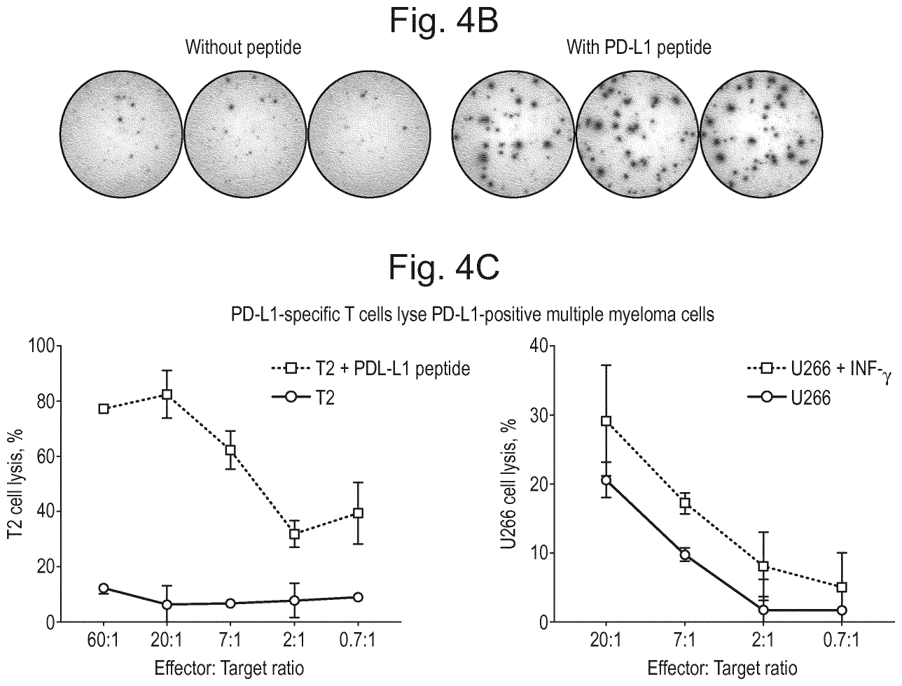

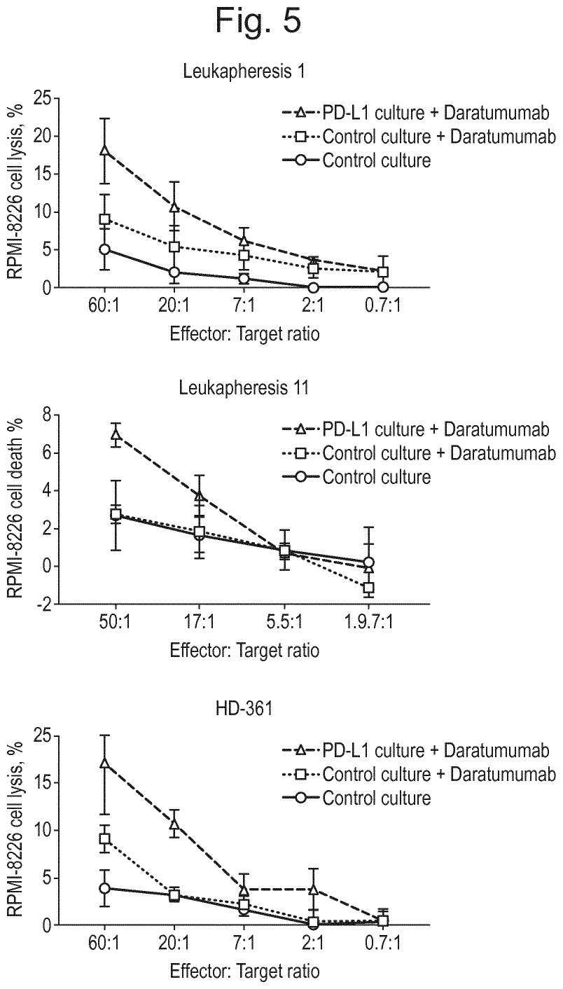

Of 20 leukapheresis products from MM patients, 19 were viable after thawing. Among the viable leukapheresis products, 7 had IFN-γ responses that were seen in triplicate against the PD-L1 peptide, meeting the definition of response in accordance with CIP and Moodie et al 2012. Due to insufficient viability, 10 samples were analyzed in duplicate experiments. Of these, 6 showed clear signs of responses without meeting the standard definition of a response due to being run in duplicate (FIG. 4A). Thus, a majority of patient samples showed spontaneous IFN-γ-responses against PD-L1.

PD-L1-specific CTLs were cytotoxic to the HLA-matched myeloma cell line U266. IFN-γ is a major inducer of PD-L1 expression and has been shown to upregulate PD-L1 on U266 cells. When U266 cells were pretreated with IFN-γ, the cells were more susceptible to being killed by the PD-L1 specific CTLs (FIG. 4B).

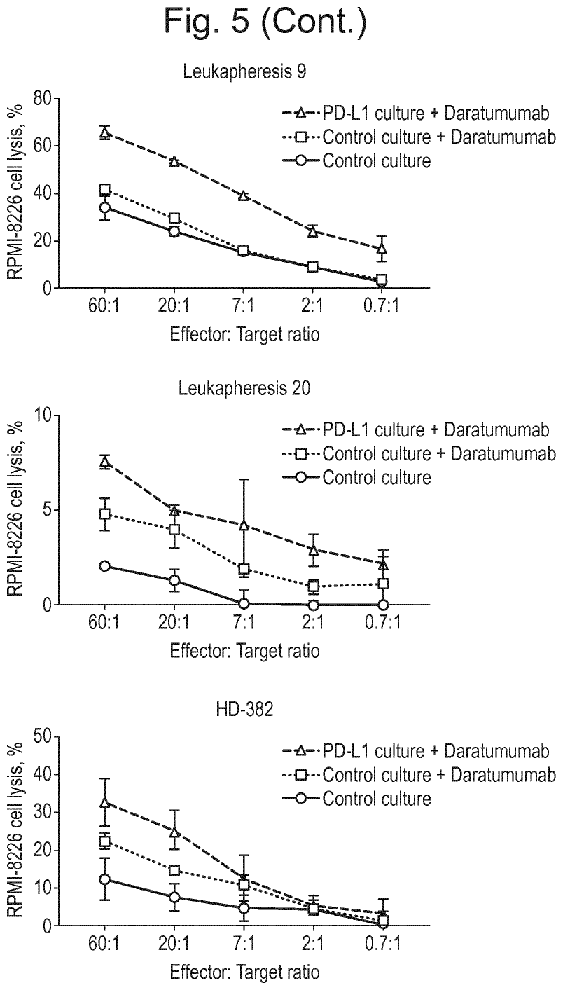

Stimulation of leukapheresis products with PD-L1 peptide and subsequent use of this culture as effector cells against RPMI-8226+/−daratumumab frequently enhanced daratumumab activity. This was seen in leukapheresis products from MM patients and in PBMCs from healthy donors (FIG. 5). Since the leukapheresis products were not HLA-matched to RPMI-8226, the largest contribution to cytotoxicity is likely attributable to NK cells, i.e. to ADCC. Interestingly, the enhancement of daratumumab activity was seen only when the leukapheresis product or HD PBMCs contained PD-L1-specific cells, as evidenced by IFN-γ ELISPOT assays against IO103 (See Example 1).

Hence, this study confirms the results in Example 1. That is, it shows that patients with myeloma harbor PD-L1-specific T cells and that MM cells are targets of cytotoxic killing by PD-L1-specific T cells. Furthermore, PD-L1-specific T cells can augment the activity of daratumumab. Together with Example 1, this is believed to be the first evidence that a peptide vaccine can boost the ADCC of a monoclonal antibody in a human model.

Given that vaccines are in general easy to administer and have very limited toxicity, a combination of an immunotherapeutic vaccine with another agent, particularly an anti-cancer antibody such as daratumumab, has significant potential.

1. A method for increasing the therapeutic benefit of an antibody to a subject, the method comprising (a) administering to said subject an immunotherapeutic composition comprising a component of an immune system checkpoint, or an immunogenic fragment of said component; and (b) also administering said antibody to the subject.

2. A method according to claim 1, wherein said checkpoint is selected from:
a) The interaction between PD1 and PDL1; or
b) The interaction between Indoleamine 2,3-dioxygenase (IDO1) or tryptophan 2,3-dioxygenase (TDO) and their substrate (tryptophan).

3. A method according to any one of the preceding claims, wherein the composition comprises an immunogenic fragment which comprises or consists of any one of the sequences in Table 1.

4. A method according to any one of the preceding claims, wherein the composition comprises an immunogenic fragment comprises or consists of any one of the sequences of SEQ ID NOs: 1, 2, 3, 4, 5, 6, 12, 14, 17, 19, 21, 48 or 49.

5. A method according to any one of the preceding claims, wherein the composition comprises an immunogenic fragment comprises or consists of any one of the sequences of SEQ ID NOs: 1, 2, or 21.

6. A method according to any one of the preceding claims, wherein said antibody is an anti-cancer antibody.

7. A method according to any one of the preceding claims, wherein said antibody is daratumumab, nivolumab, pembrolizumab, avelumab, rituximab, trastuzumab, pertuzumab, alemtuzumab, cetuximab, panitumumab, tositumomab, or ofatumumab.

8. A method according to any one of the preceding claims, wherein the composition comprises an immunogenic fragment which comprises or consists of any one of the sequences of SEQ ID NOs: 1, 2, or 21, and the antibody comprises daratumumab.

9. A method according to any one of the preceding claims, wherein steps (a) and (b) are conducted simultaneously, separately or sequentially.

10. A method according to any one of the preceding claims, which is for treating a disease in the subject, wherein the disease is susceptible of treatment by the antibody alone.

11. A method according to claim 10 wherein the disease is cancer.

12. A method for the prevention or treatment of cancer in a subject, the method comprising administering to said subject:
(i) an immunotherapeutic composition as defined in any one of the preceding claims; and
(ii) an anti-cancer antibody, such as daratumumab, nivolumab, pembrolizumab, avelumab, rituximab, trastuzumab, pertuzumab, alemtuzumab, cetuximab, panitumumab, tositumomab, or ofatumumab.

13. A method according to claim 12 wherein the composition comprises an immunogenic fragment which comprises or consists of any one of the sequences of SEQ ID NOs: 1, 2, or 21, and the antibody comprises daratumumab.
